(19) United States
(12) Patent Application Publication
(10) Pub. No.: US 2019/0143031 A1
(43) Pub. Date: May 16, 2019

(54) WEARABLE INSULIN PUMP IN A COMPACT AND REUSABLE FORM FACTOR

(71) Applicant: Richard F. ADMANI, Charlotte, NC (US)

(72) Inventor: Richard F. ADMANI, Charlotte, NC (US)

(21) Appl. No.: 15/813,494

(22) Filed: Nov. 15, 2017

(51) Int. Cl. A61M 5/142; A61M 5/145; A61M 5/168

(52) U.S. Cl. CPC A61M 5/14244; A61M 5/145; A61M 2005/31518; A61M 5/16804; A61M 5/168

(57) ABSTRACT

A wearable, adherable, tubeless, stand-alone, two-part system insulin pump includes a patch component in a first housing, wherein the patch component includes an adhesive pad, a fixed needle, a flexible tube, and collapsible reservoir connected to the flexible tube and storing insulin; and a pump component in a second housing, wherein the pump component includes a peristaltic pump configured that includes a rotor and more than one roller to pump the insulin from the flexible tube to the fixed needle and a controller for control thereof; wherein the first housing and the second housing are selectively connected to one another, and wherein the pump component is reusable with one or more pump components. Of note, the pump can support other drugs besides insulin.

### FIELD OF THE DISCLOSURE

The present disclosure generally relates to a tubeless, stand-alone, wearable, and adherable insulin pump. More particularly, the present disclosure relates to a wearable insulin pump in a compact and reusable form factor.

### BACKGROUND OF THE DISCLOSURE

Insulin pumps are medical devices which administer insulin in the treatment of diabetes. Insulin pumps can provide two types of insulin delivery—basal insulin and bolus insulin. Basal insulin is a continuous and constant delivery at a steady rate of insulin over time. The purpose of basal insulin is to cover hepatic glucose production and to maintain glucose stability during fasting states (between meals and during sleep). Basal rates are programmed to deliver a customized rate per hour, throughout the day. Bolus Insulin is delivered “on-demand,” by the patient, for food intake and/or to correct glucose levels that are above the patient's target range. Conventional carry on insulin pumps can be large and cumbersome in size as well as high cost and other pump dispose the hardware part that makes the cost very expensive. There is a need for a more compact tubeless, stand-alone, wearable and adherable insulin pump with a reusable form factor without the need of disposing the hardware parts of the pump every three days or so.

### BRIEF SUMMARY OF THE DISCLOSURE

In an exemplary embodiment, a wearable insulin pump includes a patch component in a first housing, wherein the patch component includes an adhesive pad, a fixed needle, a flexible tube, and collapsible reservoir with an injection port connected to the flexible tube and storing insulin; and a pump component in a second housing, wherein the pump component includes a peristaltic pump configured to pump the insulin from the reservoir to flexible tube the fixed needle and a controller for control thereof; wherein the first housing and the second housing are selectively connected to one another, and wherein the pump component is reusable with one or more pump components. The patch component can be attached to a user and the fixed needle inserted to delivery insulin, with the pump component attached to the patch component. The peristaltic pump can operate by pushing the insulin through the silicon tube via a rotor and one or more rollers. The peristaltic pump can be operated by a motor including one of i) a worm gear and ii) 4 gears, iii) Roller and rotor with a flat motor. The motor can be a cylinder and compact, wherein the patch component and the pump component can be less than 0.5 inches in depth, such as about 16 mm.

The motor can include a rotatable shaft that connects to the rotor on the patch component. The one or more rollers can be disposed on the rotor. The patch component can further include one or more batteries which provide power to the pump component. The pump component can further include a display screen. The pump component can further include a vibration unit configured to cause vibration alerts to a wearer. The collapsible reservoir can include an injection port where a user injects the insulin. The pump component can support a bolus delivery rate and a basal delivery rate, each adjustable. The pump component can further include a bolus button for provide a bolus delivery in addition to a continuous basal delivery. The pump component can further include a plurality of buttons for configuration and control. The pump component can be usable for about 90 days and wherein the patch component can be usable for about 3 days. The first housing can include a plurality of openings and a button for connection with a plurality of notches and a snap on the second housing. The first housing and the second housing can be sealed with one another via a sealing gasket.

In another exemplary embodiment, a wearable insulin pump kit includes a plurality of first housings each including a patch component including an adhesive pad, a fixed needle, a flexible tube, and a collapsible reservoir connected to the flexible tube and storing insulin; and a second housing including a pump component including a peristaltic pump configured to pump the insulin from the flexible tube to the fixed needle and a controller for control thereof; wherein the second housing is selectively connectable to each of the plurality of first housings and reusable with each of the plurality of first housings.

In a further exemplary embodiment, a method providing a wearable insulin pump includes providing a patch component in a first housing, wherein the patch component includes an adhesive pad, a fixed needle, a flexible tube, and a collapsible reservoir with an injection port connected to the flexible tube and storing insulin; and providing a pump component in a second housing, wherein the pump component includes a peristaltic pump configured to pump the insulin from the flexible tube to the fixed needle and a controller for control thereof; wherein the first housing and the second housing are selectively connected to one another, and wherein the pump component is reusable with one or more pump components.

### BRIEF DESCRIPTION OF THE DRAWINGS

The present disclosure is illustrated and described herein with reference to the various drawings, in which like reference numbers are used to denote like system components/method steps, as appropriate, and in which:

FIG. 1 is a top view of a pump component of a wearable insulin pump;

FIG. 2 is a bottom view of the pump component of the wearable insulin pump;

FIG. 3 is a top view of a patch component of the wearable insulin pump;

FIG. 4 is a side view of the wearable insulin 10 with the pump component connected to the patch component;

FIG. 5 is a side perspective view of an exemplary embodiment of a wearable insulin pump with both the pump component and patch component attached to one another;

FIG. 6 is a top perspective view of the wearable insulin pump of FIG. 5 with both the pump component and patch component attached to one another;

FIG. 7 is various views of the pump component of the wearable insulin pump of FIGS. 5 and 6;

FIG. 8 is various views of the patch component of the wearable insulin pump of FIGS. 5 and 6;

FIGS. 9, 10, and 11 are various perspective views of the wearable insulin pump of FIG. 5 with both the pump component and patch component attached to one another;

FIGS. 12, 13, 14, and 15 are various perspective views of the pump component of the wearable insulin pump of FIG. 5 with the front cover removed;

FIGS. 16 and 17 are various perspective views of the patch component of the wearable insulin pump of FIG. 5;

FIG. 18 is a diagram of the motor on the pump component of the wearable insulin pump of FIG. 5;

FIG. 19 is a diagram of the wearable insulin pump of FIG. 5 shown on a user;

FIGS. 20-25 are diagrams of a peristaltic pump for the wearable insulin pump of FIG. 5;

FIG. 26 is a perspective diagram of the fixed needle 86 which is inserted into the body of the wearer for drug delivery;

FIG. 27 is a perspective diagram of a cannula needle to deliver insulin or the like in the injection port;

FIGS. 28-32 are perspective diagrams of another exemplary embodiment of a wearable insulin pump;

FIGS. 33-37 are perspective diagrams of the pump on the pump component, the tube on the patch component, and the motor; and

FIGS. 38-48 are screen shots of a mobile application associated with the wearable insulin pump.

### DETAILED DESCRIPTION OF THE DISCLOSURE

In various exemplary embodiments, the present disclosure relates to a wearable insulin pump in a compact and reusable form factor. The objective of the wearable insulin pump described herein is a compact form that can be worn all the time easily and a partially reusable design to provide lower costs. The wearable insulin pump is wearable (e.g., via an adhesive), tubeless, and in multiple parts (e.g., a patch component and a pump component). The wearable insulin pump includes an adherable peristaltic pump to deliver an injectable drug (e.g., insulin or any other injectable drug) via subcutaneous injection. Note, while the wearable insulin pump is described herein with reference to insulin, those skilled in the art will appreciate other drugs are also contemplated such as, without limitation, pain management drugs, cancer treatment drugs, chemo therapy, and the like. The patch component is designed for use for a few days (e.g., 3 days) and the pump component is configured to attached to the patch component and to operate over a longer time period (e.g., months). In this manner, the patch component can be replaced periodically with the same pump component used to lower costs. For example, the pump component can “snap” on the adhesively connected patch component. Advantageously, the peristaltic pump in the pump component is extremely compact, simple and convenient to install and operate, etc. The pump provides the benefits of continuous subcutaneous insulin infusion therapy and allows the user to delivery insulin on demand at mealtimes. The wearable insulin pump features a small, lightweight, self-adhesive disposable tubeless device. The system uses two-part system—patch (Disposable part) to be worn for three days or so and the pump is reusable for 90 days, etc.

The wearable insulin pump is wearable directly on a user's body through an adhesive, i.e., the wearable insulin pump is adherable to the body. Of course, conventional insulin pumps can be wearable in the sense they are attached to a belt or in a pocket. However, the wearable insulin pump described herein is directly attached to the user's body. Also, the wearable insulin pump is a stand-alone pump and does not require a remote-control device or a smartphone for control thereof. Of note, there are existing wearable and tubeless pumps, however the wearable insulin pump includes a two-part approach to minimize waste and cost, i.e., an adherable part that is disposed every few days and a pump which has a much longer lifetime. Also, the existing wearable and tubeless pumps are not stand alone and require a remote-control device such as a smart phone or the like.

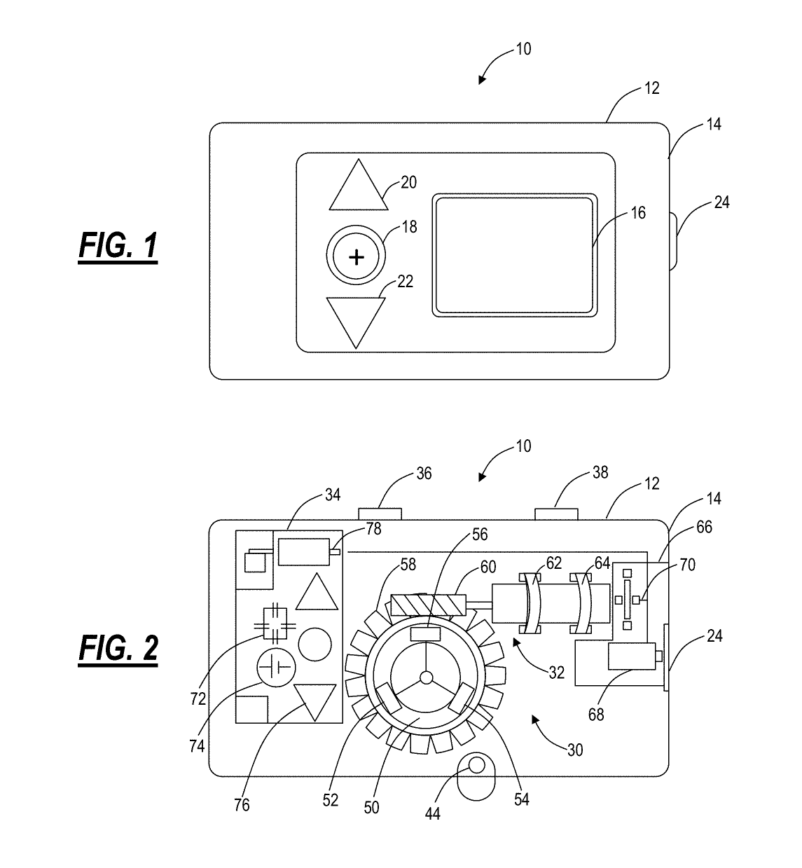

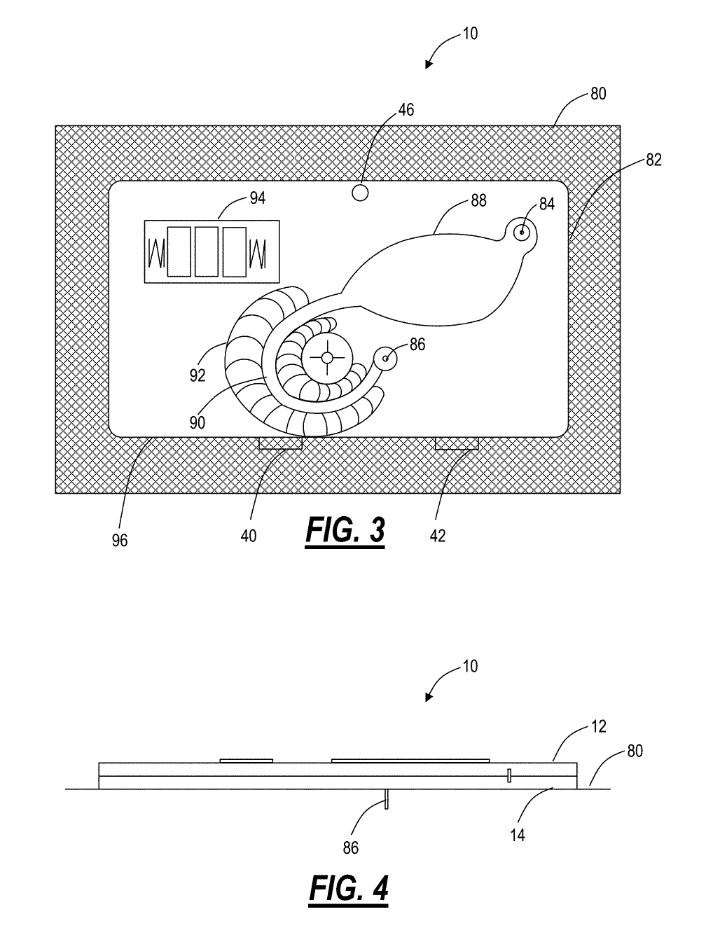

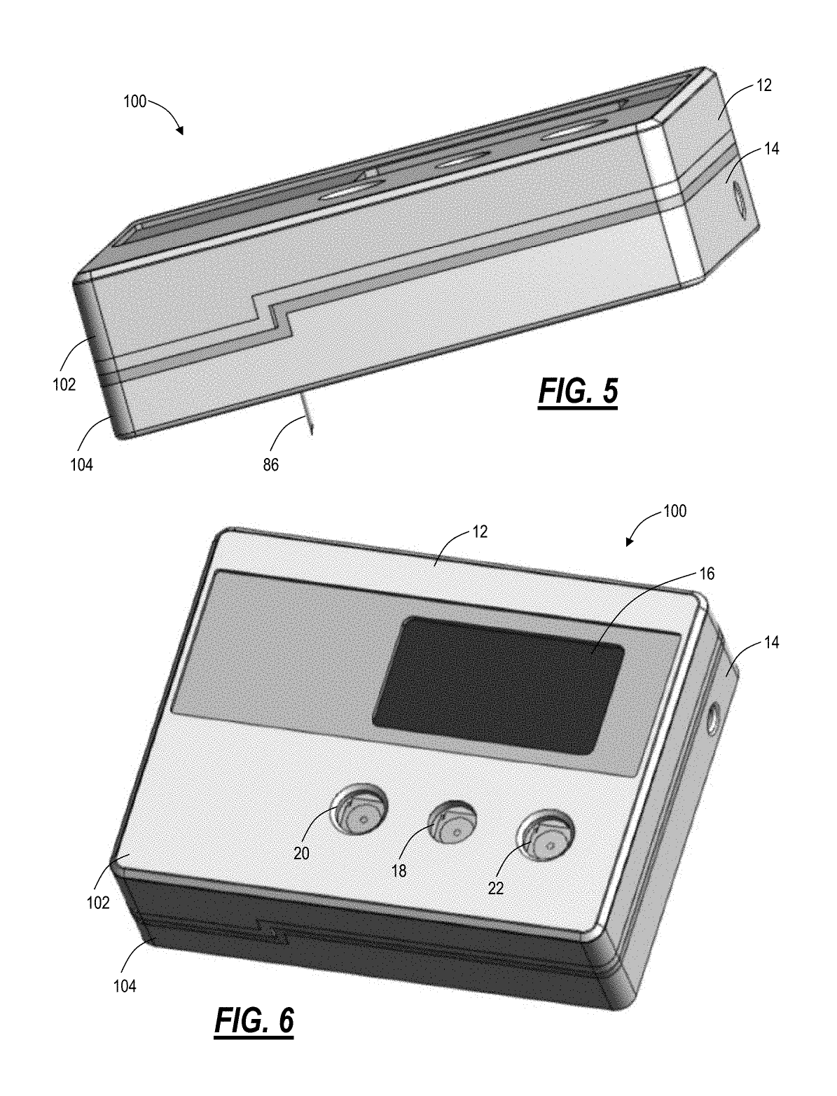

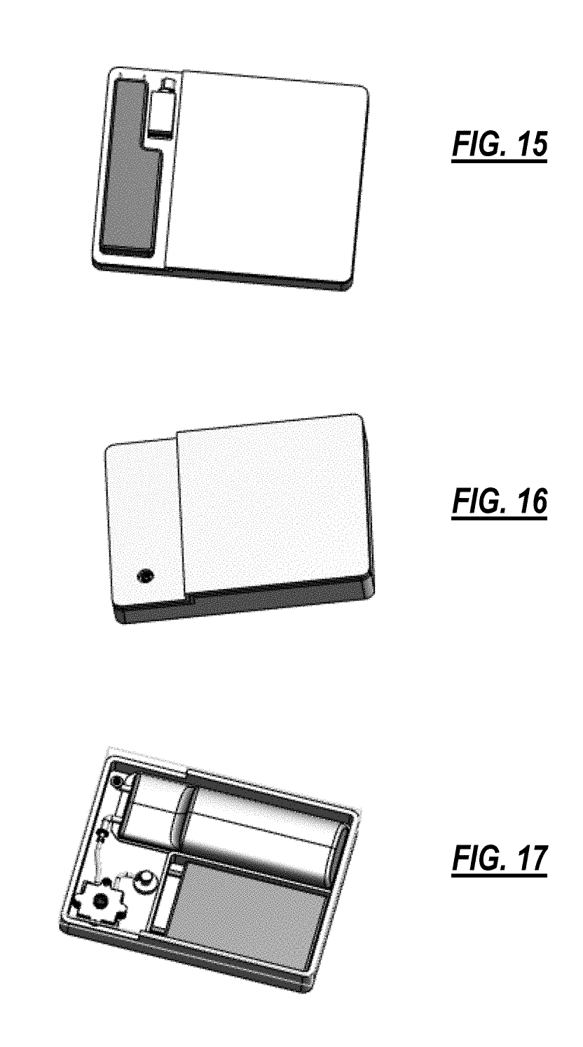

Referring to FIGS. 1-4, in exemplary embodiments, various diagrams illustrate a wearable insulin pump 10 in accordance with the present disclosure. Specifically, FIG. 1 is a top view of a pump component 12 of the wearable insulin pump 10. FIG. 2 is a bottom view of the pump component of the wearable insulin pump 10. FIG. 3 is a top view of a patch component 14 of the wearable insulin pump 10. FIG. 4 is a side view of the wearable insulin pump 10 with the pump component 12 connected to the patch component 14. Another variation of the wearable insulin pump 10 is the wearable insulin pump 100 illustrated in FIGS. 5-19. Note, the wearable insulin pumps 10, 100 have similar characteristics.

The wearable insulin pump 10 is designed to be worn with the patch component 14 adhesively connected to a wearer and the pump component 12 snapped on top of the patch component 14. Again, the wearable insulin pump 10 has an extremely compact design, such as 2.0″ (L)×1.5″ (H)×0.5″ (D). In FIG. 1, the top of the pump component 12 is visible to the wearer. The pump component 12 includes a housing 14 such as plastic or the like, a display screen 16, an on/off button 18, a scroll up button 20, a scroll down button 22, and a bolus button 24. Note, the bolus bottom 24 can also be located near the buttons 18, 20, 22. The wearer (user) can interact and control the wearable insulin pump 10 through the top of the pump component 12. The scroll up button 20 and the scroll down button 22 can be used to interact with the display screen 16, change doses of the basal (daily rate), set the date and time, set the amount of insulin injected into the reservoir, etc. Of note, the bolus button 24 is integrated into the electronics on the wearable insulin pump 10, 100, i.e., the bolus button 24 is not all mechanical and use of the bolus button 24 can be linked to alerts, displays, communications, etc.

In an exemplary embodiment, there are no settings with respect to the bolus. That is, the bolus button 24 delivers one unit of insulin (e.g., 10 uL). So, if the patient needs five units of insulin for a meal, the patient would press the bolus button 24 five times, or if they need 10 Units they press 10 time on demand during food intake, etc. The bolus button 24 allows the wearer to initiate a bolus dose. The on/off button 18 can support power on, power off, start, confirm, prime, suspend, resume, etc. That is, the on/off button 18 can be used in coordination with the scroll up button 20 and the scroll down button 22 for control of the wearable insulin pump 10.

In FIG. 2, the bottom of the pump component 12 is shown and this bottom part engages the patch component 14 (shown in FIG. 3). The pump component 12 includes the housing 14, a pump 30, a motor 32, and a controller system 34. The housing 14 can include notches 36, 38 which engage openings 40, 42 in the patch component 14 and a fastener 44 which locks to a snap button 46 on the patch component 14. Thus, to connect the pump component 12 to the patch component 14, the notches 36, 38 are placed in the openings 40, 42 and the fastener 44 is snapped on the snap button 46 selectively locking the pump component 12 to the patch component 14.

The pump 30 is a peristaltic pump including a rotor 50 with three rollers 52, 54, 56 and gears 58 which engage a worm wheel 60 connected to the motor 32. The motor 32 is physically held in place in the pump component 12, such as through straps 62, 64 or the like. The motor 32 is connected to motor control circuitry 66 for control thereof. Note, the rotor 50 is shown in the pump component 12, but in another exemplary embodiment (in the wearable insulin pump 100 described herein), the rotor 50 can be in the patch component 14. Rotation of the worm wheel 60 causes corresponding rotation of the rotor 50 and the rollers 52, 54, 56 via the gears 58. The motor control circuitry 66 can include a bolus button control 68 which is configured to detect the bolus button 24 being pushed and circuitry 70 for power and control of the motor 32. The bolus button control 68 and the circuitry 70 is connected to the controller system 34. There can also be an encoder on the rotor 50 to control the position of the rotation.

The motor 32 can use a worm gear or any other flat type motor to rotate the rotor 50 and the rollers 52, 54, 56 using a long cylinder motor, e.g., brushless DC motor, brush DC motor, stepper DC motor, etc. The motor 32 can be flat and the wearable insulin pump 10 uses the flat motor with attached gears, gearbox, etc. to connect the motor shaft to the rotor 50.

The controller system 34 can include a controller 72 (e.g., a Printed Circuit Board Assembly (PCBA) controller), a power unit 74, corresponding control points 76 for the on/off button 18, the scroll up button 20, and the scroll down button 22, and a vibration unit 78. The controller 72 can provide control of the wearable insulin pump 10 based on feedback from the various buttons as well as control of the display unit 16. The power unit 74 can provide power to the various components in the pump component 12. In an exemplary embodiment, the power unit 74 can include one or more batteries. In another exemplary embodiment, the power unit 74 can include a connection to corresponding batteries in the patch component 14, such as a super capacitor.

The wearable insulin pump 10 can include two batteries—batteries 94 in the patch component 14 which are disposable and used to drive the pump, the display 16, and the motor 32 for the life of the patch component 14 (e.g., 3 days, etc.), and a battery such as part of the power unit 74 that saves the settings on the pump component 12 (e.g., date and time, basal and bolus delivery rates, etc.). Other embodiments are contemplated including rechargeable batteries, self-powered or recharging batteries such as capturing motion of a wearer and converting the motion into power. Also, the batteries can be in either the pump component 12, the patch component 14, or both. The vibration unit 78 is configured to vibrate to provide sensory feedback to the wearer.

The wearable insulin pump 10 can also include a communications interface such as on the pump component 14 for external communications. The communications interface can include Bluetooth, Wi-Fi, Bluetooth Low Energy, or some other wireless protocol. The communications interface can communicate with a smart phone and an associated application (“app”). All the data collected basal/bolus can be transferred to the app so the user has a data for the delivery rate, and this data can be uploaded to the cloud from the smart phone so a doctor can access the cloud and learn on the basal and bolus rate with their glucose reading so can adjust medicine, insulin delivery rate and other, this is the only way doctor can help patient if they gather these data, now patient never log these data to share with the doctor and the doctor guess on adjusting the meds.

Advantageously, no current solutions include the display screen 16 on the top of a wearable device. Conventional approaches rely on a separate, connected controller/receiver.

In FIG. 3, the top of the patch component 14 is shown and this top part engages the pump component, via the openings 40, 42 and the snap button 46. The patch component 14 includes an adhesive pad 80 connected a housing 82. The adhesive pad 80 is configured to hold the wearable insulin pump 10 via the patch component 14 to the wearer. The housing 82 (e.g., plastic of the like) connects to the housing 14 to form the wearable insulin pump 10 in two parts.

The patch component 14 includes an insulin injection port 84, a fixed needle 86, a collapsible reservoir 88, a silicon tube 90 extending from the collapsible reservoir 88 to the fixed needle 86, and a base/molding 92 surrounding the silicon tube 90. The silicone tube 90 is placed and held in a cavity channel molded with the base of the disposable part, i.e., the base/molding 92. In the wearable insulin pump 100, the injection port 84 is on the bottom part of the base of the patch component 14, next to the needle 86.

The patch component 14 can also include batteries 94 which can connect to the power unit 74. The housing 82 can also include a sealing gasket 96 which forms a waterproof seal between the patch component 14 and the pump component 12 when engaged.

The adhesive pad 80 is connected to the housing 82 and holds the patch component 14 in place on the wearer. For example, the patch component 14 can be placed near a port on the wearer where the fixed needle 86 is inserted. The adhesive pad 80 is strong enough to hold both the wearable insulin pump 10, 100 in place on the wearer. Further, with the sealing gasket 96, the wearable insulin pump 10 can be worn anywhere (swimming, in the shower, etc. IPX7 (water proof standard)).

The collapsible reservoir 88 can include the injectable drug, e.g., insulin, and can have a capacity of around 3 ml or the like. The fixed needle 86 can be a 31G fixed needle with 4 mm in length. The fixed needle 86 sticks out of the base of the patch component 14, such as at 90 deg., and can include a removable cover during transport. The makes the design simple and low cost as other approaches require insertable needles. The patch component 14 can be sterilized by Ethylene oxide sterilization.

The batteries 94 can be removable, held in place with battery clips and a connection leaf spring. Alternatively, the batteries 94 can be fixed and disposed when the patch component 14 is disposed. The battery also does not to be removed, e.g., it can be used for 3 days and after that its will be disposed with the patch component 14.

The wearable insulin pump 10 is illustrated as a two-part system with the pump component 12 and the patch component 14. The patch component 14 is frequently disposable such as every 3 days, (for insulin 3 days, other drugs can have different frequencies) preferably every 3 days. Note, insulin, as the injectable drug, the injectable location needs to be moved every 3 days or so. The pump component 12 is configured to attach/detach from the patch component 14 and is configured to last considerably longer, such as months, preferably about 3 months. In this manner, the reusable components are leverage in the pump component 12 to keep costs down whereas the disposable components (needle 86, drug in the collapsible reservoir 88, the batteries 94, etc.) are in the patch component 14.

An important aspect of the wearable insulin pump 10, 100 is the use of the peristaltic pump 30. There are three exemplary operations for the pump 30. First, the rollers 52, 54, 56 can be on the side of the rotor 50 to squeeze the tube 90 between the rollers 52, 54, 56 and the side walls (the base/molding 92) to drive the liquid (i.e., insulin or the like). Second, the rotor 50 can include the rollers 52, 54, 56 on its surface and a top cover over the rotor 50 can create pressure (squeeze) on the tube 50 to drive the liquid (i.e., insulin or the like) through the tube 90. Third, the rotor 50 and the rollers 52, 54, 56 can press on the tube 90 from any direction including the bottom to drive the liquid.

FIG. 4 illustrates a side view of the wearable insulin pump 10 with the pump component 14 engaged to the patch component 12. As shown, the wearable insulin pump 10 is extremely compact and lightweight.

The wearable insulin pump 10 utilizes a peristaltic pump between the pump component 12 and the patch component 14. A peristaltic pump is a type of positive displacement pump used for pumping a variety of fluids. In the wearable insulin pump 10, the fluid (injectable drug such as insulin) is contained within the silicon tube 90 and the collapsible reservoir 88 fitted inside a circular pump casing, i.e., the base/molding 92. The rotor 50 with the three rollers 52, 54, 56 attached to the external circumference of the rotor 50 compresses the silicon tube 90. The rollers 52, 54, 56 can also be referred to as shoes, wipers, lobes, etc. and there can be more or less than three.

The collapsible reservoir 88 expands/contracts based on the amount of insulin therein. Advantageously, this approach reduces the amount of pressure required for the pump 30 to push the insulin, allowing a smaller, more compact motor 34.

As the rotor 50 turns, the part of the silicon tube 90 under compression is pinched closed (or “occludes”) thus forcing the fluid to be pumped to move through the silicon tube 90. Additionally, as the silicon tube 90 opens to its natural state after the passing of the cam (“restitution” or “resilience”) fluid flow is induced to the pump. This process is called peristalsis and is used in many biological systems such as the gastrointestinal tract. Typically, there will be two or more rollers 52, 54, 56 (of note, there can also be just one roller), or wipers, occluding the silicon tube 90 (note, other materials besides silicon are also contemplated), trapping between them a body of fluid. The body of fluid is then transported, at ambient pressure, toward the pump outlet, i.e., the fixed needle 86. Peristaltic pumps may run continuously, or they may be indexed through partial revolutions to deliver smaller amounts of fluid.

The wearable insulin pump 10 contemplates various approaches to pinching the silicon tube 90 to deliver the injectable drug. In the example of FIGS. 1-3, the pinching from the rollers 52, 54, 56 is from the top. However, other embodiments are contemplated such as from the side or bottom.

The wearable insulin pump 10 can support two types of delivery rates—basal and bolus. The basal delivery rate is a constant and continuous delivery of insulin at a very small amount to keep the wearer's glucose level in control. The bolus delivery is the delivery of insulin on demand at meal time (e.g., breakfast, lunch, dinner, snack). or as needed to lower blood sugar level. Every time the pump cycles for either basal or bolus delivery it can deliver 1 uL of Insulin (0.1 U of Insulin). In a basal set up, the user sets the pump with a basal rate (at time of setting), the set rate is fixed and does not change. For example, if the user set the pump for a Basal rate of 24 Unit of Insulin in a day (240 uL per 24 hours), then the pump to deliver as follows: 240 uL/24 hrs=10 uL per hour (that means the pump will cycle once every 6 minutes by delivering 1 uL.) The pump will cycle 10 times an hour once every 6 minutes. For the bolus delivery, the user press on the bolus button and every time that happens the pump to cycle 10 times (1 uL×10 times)=10 uL (1.0 Unit of Insulin).

The display screen 16 can be a black Light Emitting Diode (LED) screen with a white lid digital display. The display screen 16 with the controller system 34 can continuously display information such as date and time, expiration date and time of the patch component 14, insulin volume in the collapsible reservoir 88, pump status is suspended, basal rate (e.g., daily rate), pump status (e.g., alarms and alerts), etc. The display is also for setting.

In an exemplary embodiment, the controller system 34 can include a wireless interface (e.g., Bluetooth or some other Personal Wireless Area Network) to connect to a smart phone. For example, the smart phone (e.g., iPhone, Android, etc.) can execute an application for control and status of the wearable insulin pump 10. This is to transfer the data to the application in the phone and allow the patient to view on their smartphone application or in the cloud.

In operation, first the patch component 14 is attached to the wearer and then the pump component 12 is attached to the patch component 14. The wearer or the like can press the on/off button 18 (e.g., for a predetermined time period such as 2 s) to turn the wearable insulin pump 10 on.

The wearer can set the date/time on the display screen 16. For example, the display screen 16 can show a Month/Day/Year with each blinking so the wearer can scroll up/down (with the buttons 20, 22) to set appropriately. Also, the pump component 12 can include some on-board power reserve in the power system 72 to keep the date/time and other data as the pump component 12 is attached to different patch components 14. For example, the pump component 12 may only require setting the date/time once and it lasts for about 100 days, which is about the lifetime of the pump component 12.

In an exemplary embodiment, the patch component 14 can come with the insulin in the collapsible reservoir 88. In another exemplary embodiment, the user can inject insulin into the collapsible reservoir 88 via the insulin injection port 84.

For example, after setting the date and time, the user measures the amount of insulin via a syringe and injects the insulin into the collapsible reservoir 88. The user can then turn on the wearable insulin pump 10 by pressing and holding the on/off button 18. The user can push the confirm button (e.g., the on/off button 18) to accept and jump to Day, Year, AM or PM, Hour, minute by pressing confirm button to move from one value to the other value. The operator can change any of the blinking valve before confirming by pressing the up or down scroll buttons 20, 22. If the operator confirms any of the value and wants to go back to change the saved value just press on the scroll down button.

Next, the wearer is prompted for entering the insulin volume for injection. For example, the default full value can be 200 units—this can be modified as needed. The user can press the scroll button (up or down) to select the value and press the confirm button to move to the daily basal rate. The daily basal rate can be set and can be defaulted to 10 units. The scroll buttons 20, 22 can be used=to increase or decrease the daily rate by increments of 1.0 unit. After confirming the basal rate, the screen moves to Priming.

The user holds the on/off button 18 to start priming the tube line, as soon as the droplets of insulin start coming out of the fixed needle 86, the user can release the on/off button 18 to stop priming. The user can then take the liner off the adhesive pad 80 and place by pushing the patch component 14 onto the injection area.

The user can press the on/off button 18 to start the basal delivery for 3 days of therapy (72 hours). The pump 30 allows the user to extend the use of the pump from 72 hours to 80 hours and then it turns off the pump 30.

The user can suspend the basal delivery by holding down the on/off button 18 for 2 second. After the pump 30 suspends the delivery, the wearable insulin pump 10 vibrates once to confirm the pump 30 is in the suspended mode. The display screen 16 shows “Pump Suspended” or “Delivery Off,” the user can resume the pump by holding the on/off button 18 for 2 seconds. The wearable insulin pump 10 vibrates once to confirm to the user the pump 30 is resumed.

Every time the wearer wants to deliver bolus insulin on demand, the wearer can hold the bolus button for 2 seconds or the like, the wearable insulin pump 10 vibrates once to confirm to the user that the pump 30 dispensed 1 unit of insulin. Every time the user press on the bolus button, the pump 30 will deliver 1 unit of insulin (10 uL). If the wearer needs 6 units of insulin (60 uL) then the user pressed the bolus button for 6 times by holding for 2 second each time.

If the user wishes to shut down the wearable insulin pump 10, the user can hold the on/off button 18 for 5 seconds and then the wearable insulin pump 10 vibrates and confirms to the user that the wearable insulin pump 10 is shutting down.

The wearable insulin pump 10 can support various alarms which can be displayed on the display screen 16 or indicated by the vibration unit 78. For example, the following are examples of alarms/alerts:

| | |
|---|---|
| Occlusion | The wearable insulin pump 10 continues vibrating |
| | every 10 seconds until the user shut down the pump. |
| Empty reservoir | The wearable insulin pump 10 continues vibrating |
| | every 10 seconds until the user shut down the pump. |
| Pump failure | The wearable insulin pump 10 continues vibrating |
| | every 10 seconds until the user shut down the pump. |
| Low battery | The wearable insulin pump 10 continues vibrating |
| | every 10 seconds until the user shut down the pump. |
| Replace patch | The wearable insulin pump 10 continues vibrating |
| | every 10 seconds until the user shut down the pump |
| | and replaces the patch component 14. |
| Replace pump | The wearable insulin pump 10 continues vibrating |
| | every 10 seconds until the user shut down the pump |
| | and replaces the pump component 12. |
| Low reservoir | The wearable insulin pump 10 to vibrate every 3 |
| | seconds for 5 times only. |
| Expired Patch at 72 | The wearable insulin pump 10 to vibrate every 3 |
| hours | seconds for 5 times only. |
| Bolus delivery | Every Bolus delivery the wearable insulin pump 10 |
| | to vibrate once. |
| Suspended/Resumed | Every time the user suspends/resumes the wearable |
| | insulin pump 10, the wearable insulin pump 10 to |
| | vibrate once |

Also, all of the above alarms/alerts can be displayed on the display screen 16, e.g., Pump Suspended, Occlusion, Empty reservoir, low reservoir, Pump failure, Low battery, expired, Patch (72 hours), Replace Patch (80 hours), expired Pump (after 100 days).

Again, the wearable insulin pump 10 is compact and fully integrated. Traditional insulin pumps require the pump to be worn and clipped on the belt or in the pocket. Other wearable devices remove this needed for the external pump, but lack reusability and the presence of the integrated display screen 16. Other advantages of the wearable insulin pump 10 include the on demand bolus button 24, the on/off button 18 for delivery suspension, vibrations for alerts, etc.

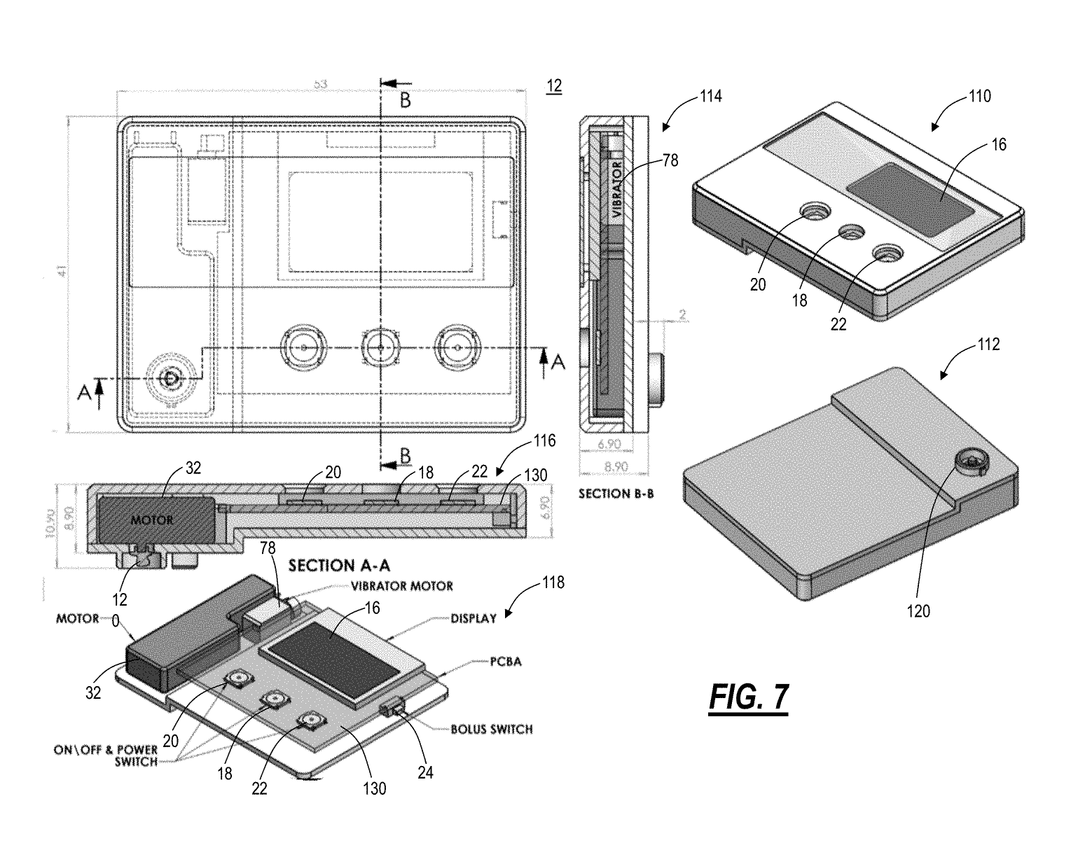

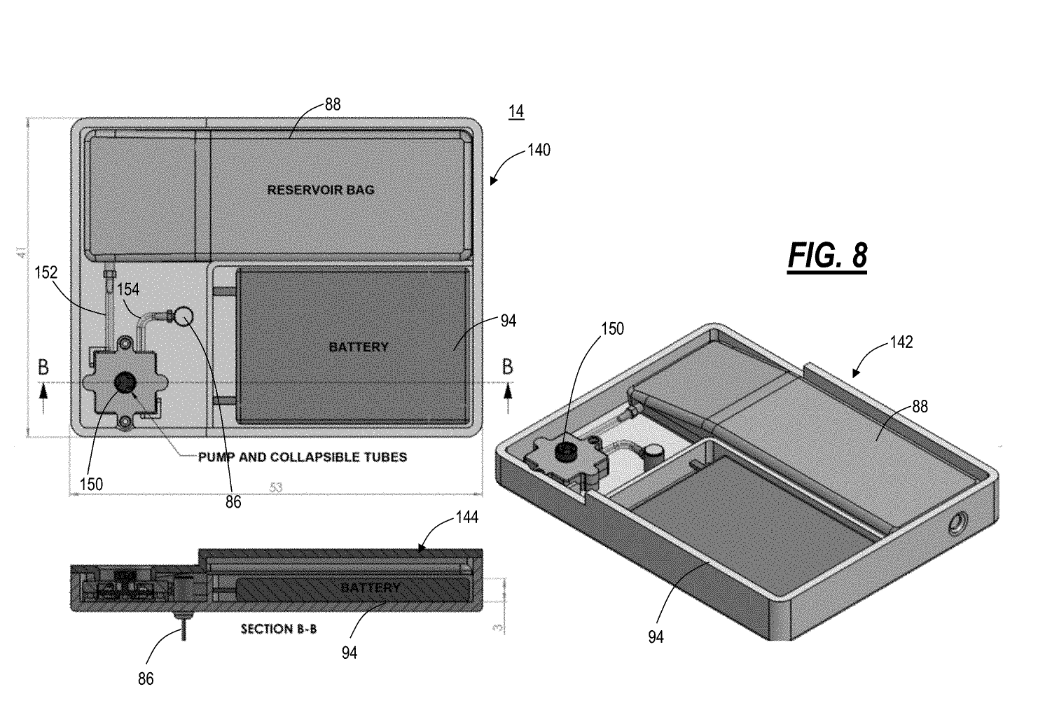

Referring to FIGS. 5-8, in an exemplary embodiment, various diagrams illustrate another exemplary embodiment of a wearable insulin pump 100 in accordance with the present disclosure. Specifically, FIG. 5 is a side perspective view of an exemplary embodiment of the wearable insulin pump 100 with both a pump component 12 and a patch component 14 attached to one another. FIG. 6 is a top perspective view of the wearable insulin pump 100 with both the pump component 12 and the patch component 14 attached to one another. FIG. 7 is various views of the pump component 12 and FIG. 8 is various views of the patch component 14. Note, the wearable insulin pump 100 is similar to the wearable insulin pump 10.

FIGS. 5 and 6 illustrate the assembled, connected wearable insulin pump 100. The pump component 12 and the pump component 14 each include a housing 102, 104, respectively. The housings 102, 104 have a substantially rectangular shape. The housings 102, 104 have a complementary shape which when connected to one another forms a larger rectangular shape. Of course, other embodiments are contemplated.

FIG. 7 includes various views of the pump component 12 for the wearable insulin pump 100. Specifically, FIG. 7 includes a top view 110, a bottom view 112, cross-sectional views 114, 116, and a component view 118. The housing 102 includes the display 16, the on/off button 18, a scroll up button 20, a scroll down button 22, or similar. The bottom of the housing 102 includes a shaft 120 which extends out of the housing 102 for connection to associated components in the patch component 14.

The interior components of the pump component 12 include a Printed Circuit Board Assembly (PCBA) 130 with the display 16 attached, the buttons 18, 20, 22, the bolus button 24, associated electronics, and connections to the motor 32 and the vibration unit 78. The motor 32 is a self-contained unit and configured to rotate the shaft 120 which connects to gears and rotors in the patch component 14. As described herein, the motor 32 can include a worm gear, 4 worm gears, rollers and rotor or the like.

FIG. 8 includes various view of the patch component 14 for the wearable insulin pump 100. Specifically, FIG. 8 includes a top view 140, a top perspective view 142, and a cross-sectional view 144. The patch component 14 includes the collapsible reservoir 88 which connects to a pump 150 via a tube 152 and the pump 150 connect to the needle 86 via another tube 154. The pump 150 can include gears and/or rotors which perform peristaltic pumping based on rotation of the shaft 120. In the wearable insulin pump 100, the last gear connecting to other gears with the motor 32 is on the patch component 14. Here, the pump 150 can connect to the shaft 120 that connects to a rotor on the patch component 14 to drive the rotor and then drive the liquid through the tubes 152, 154.

The various portions of the tubes 152, 154 can be injected, two-part silicon that are welded together to create the shape of the tubes 152, 154. Also, other material types for the tubes 152, 154 are also contemplated, e.g., rubber or any other elastic material.

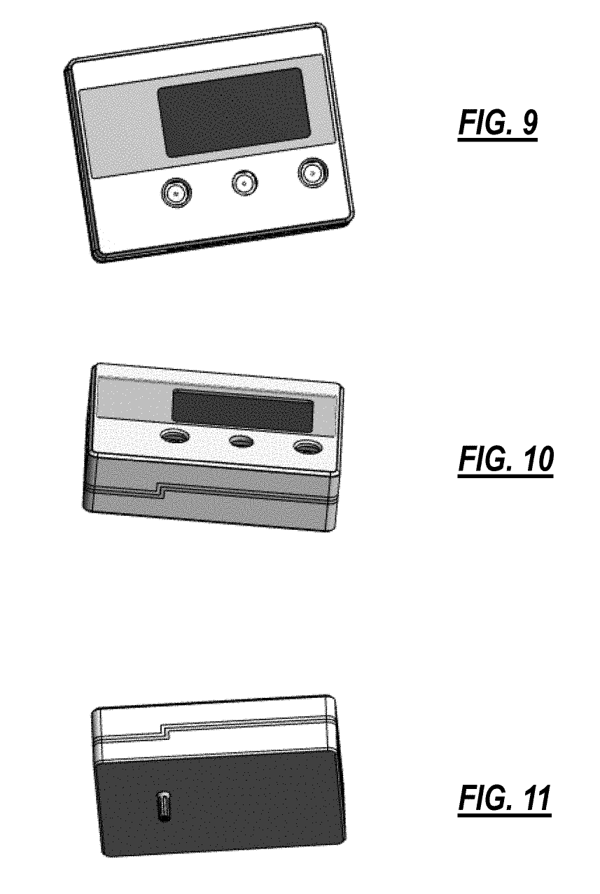

FIGS. 9, 10, and 11 are various perspective views of the wearable insulin pump 100 with both the pump component 12 and the patch component 14 attached to one another.

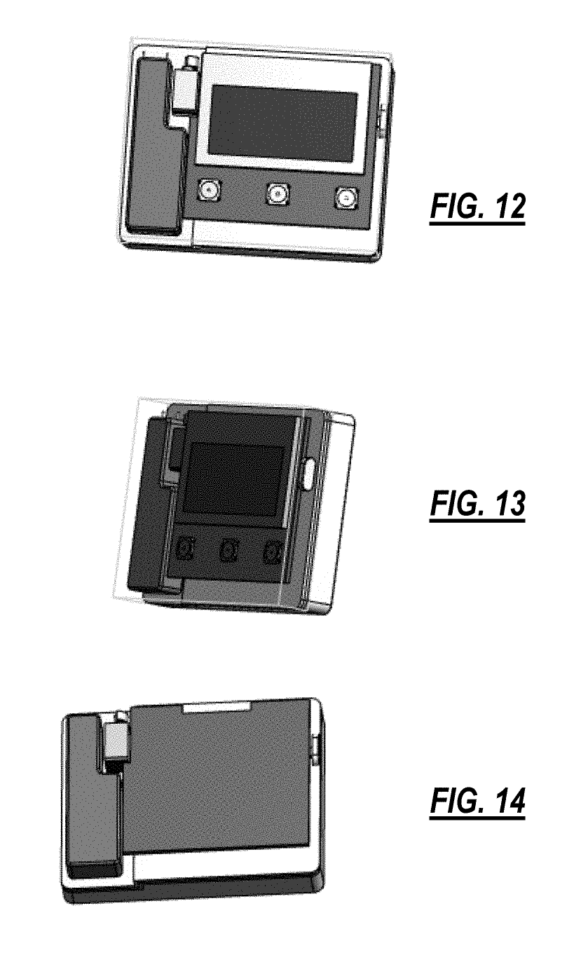

FIGS. 12, 13, 14, and 15 are various perspective views of the pump component 12 of the wearable insulin pump 100 with the front cover removed.

FIGS. 16 and 17 are various perspective views of the patch component 14 of the wearable insulin pump 100.

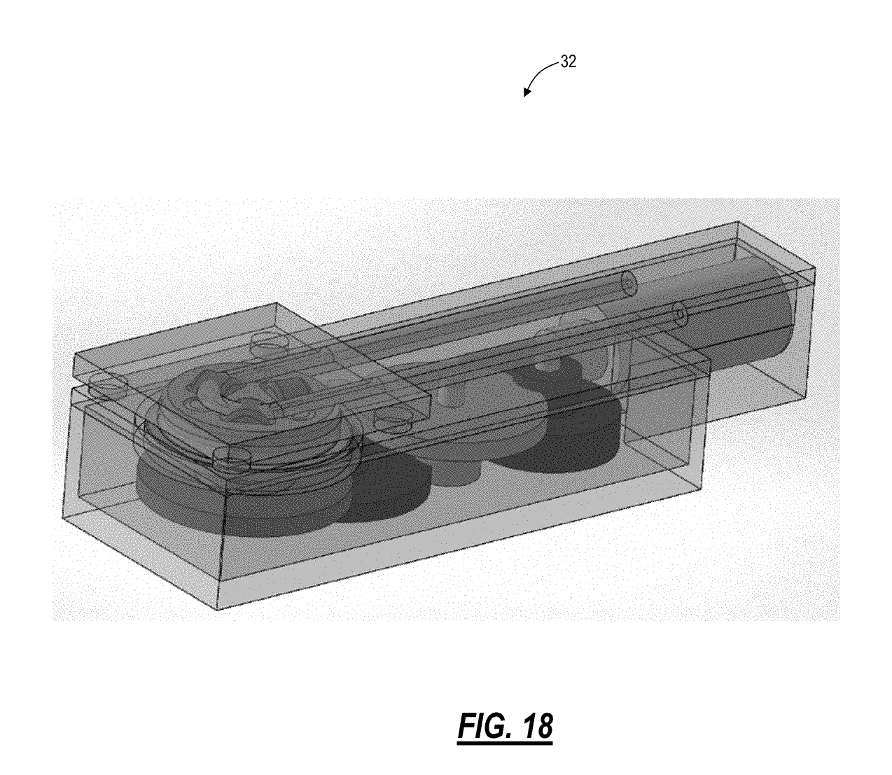

FIG. 18 is a diagram of the motor 32 on the pump component 12 of the wearable insulin pump 100. Specifically, the motor 32 includes a plurality of gears which are rotated by a cylinder motor. Overall, the motor 32 has a compact, flat design. The gears connect to the shaft 120 which is configured to rotate a gear in the pump 150 on the patch component 14.

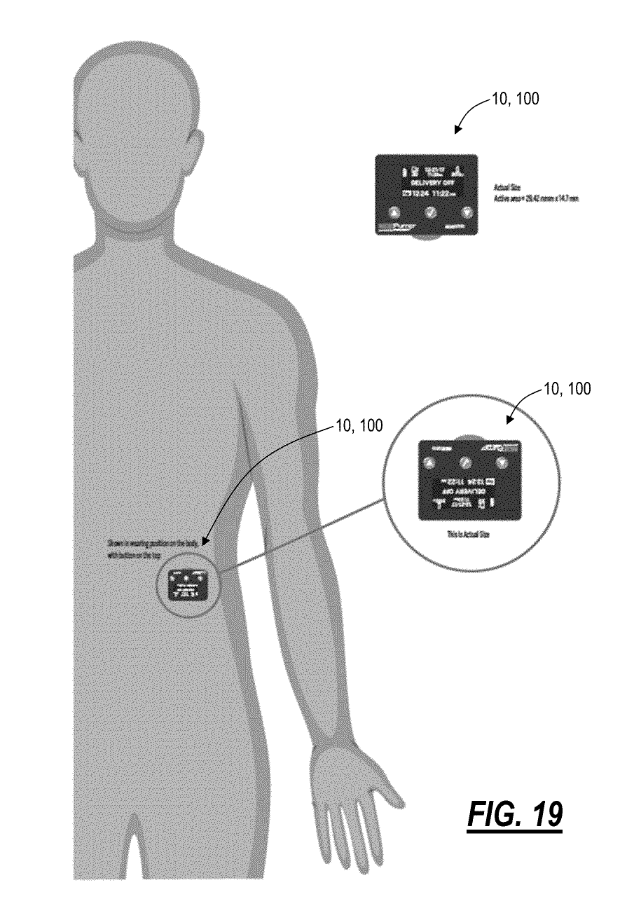

FIG. 19 is a diagram of the wearable insulin pump 100 shown on a user.

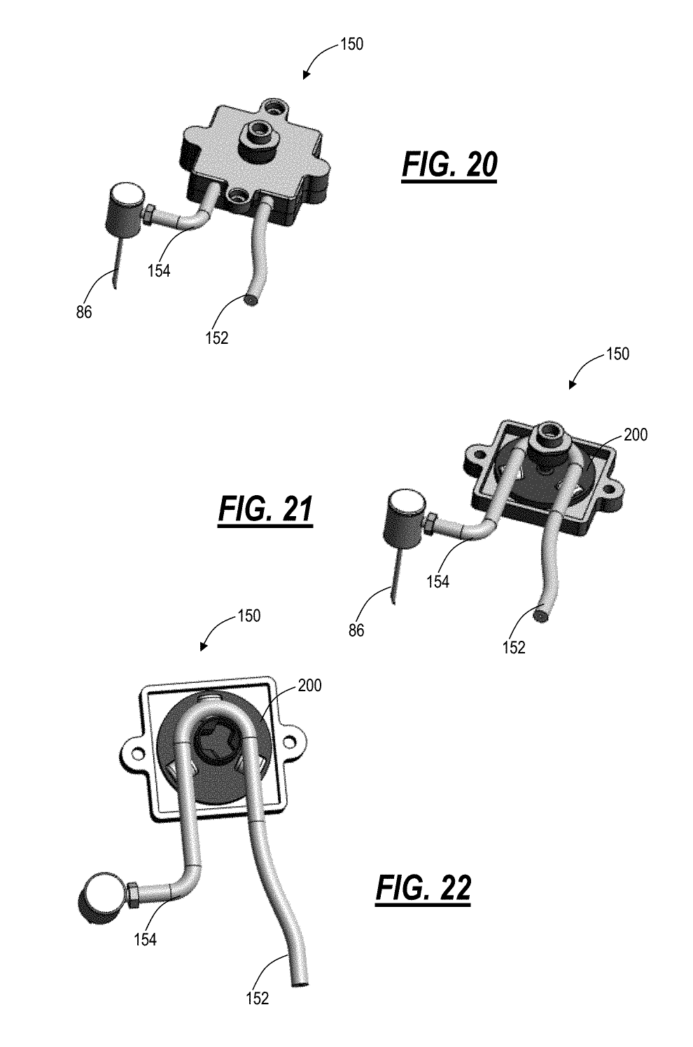

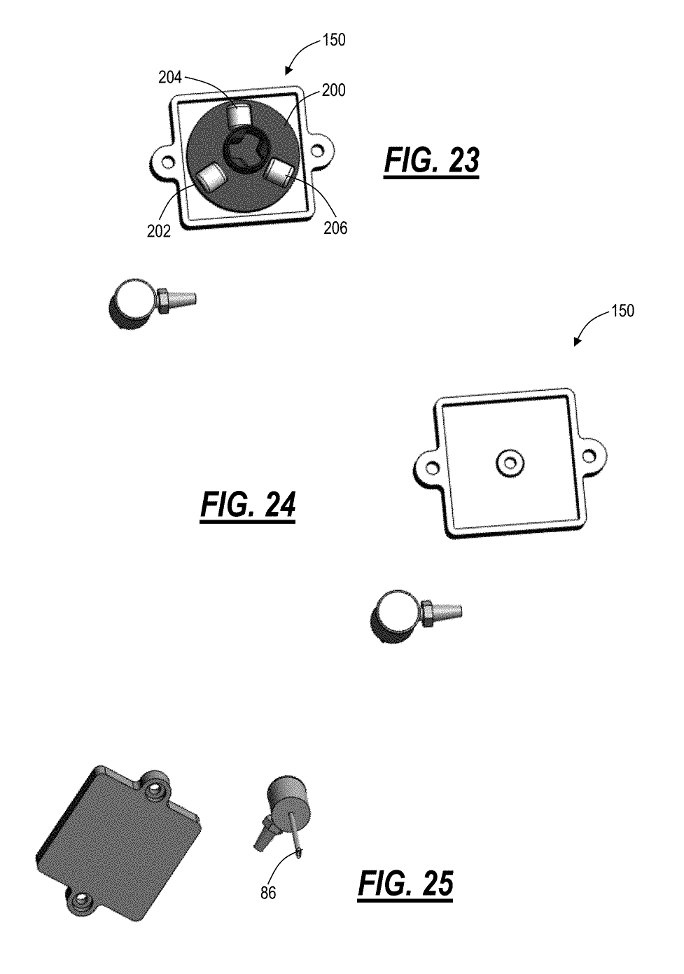

FIGS. 20-25 are diagrams of a peristaltic pump 150 for the wearable insulin pump of FIG. 5. Again, the shaft 120 from the motor 32 connects to the pump 150 to a rotor 200 which includes one or more rollers 202, 204, 206 attached thereto. The shaft 120 turns the rotor 200 which causes pressure from the rollers 202, 204, 206 over the tube 152, 154 driving the liquid from the collapsible reservoir 88 to the needle 86.

An important aspect of the wearable insulin pump 10, 100 is the use of the peristaltic pump 30. There are three exemplary operations for the pump 30, 150. First, the rollers 52, 54, 56. 202, 204, 206 can be on the side of the rotor 50 to squeeze the tube 90 between the rollers 52, 54, 56 and the side walls (the base/molding 92) to drive the liquid (i.e., insulin or the like). Second, the rotor 50 can include the rollers 52, 54, 56 on its surface and a top cover over the rotor 50 can create pressure (squeeze) on the tube 50 to drive the liquid (i.e., insulin or the like) through the tube 90. Third, the rotor 50 and the rollers 52, 54, 56 can press on the tube 90 from any direction including the bottom to drive the liquid.

The peristaltic pump 150 delivers insulin or the like by rotating the rotor 50 360 degrees and stopping the motor in the proper position by a sensor (encoder). Also, the motor could stop the rotation within a time period (e.g., run the motor for 1.8 sec and then stop).

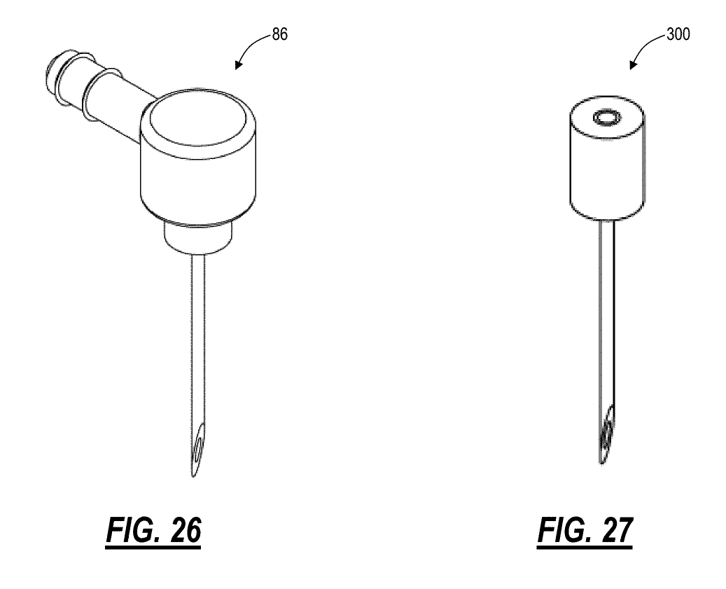

FIG. 26 is a perspective diagram of the fixed needle 86 which is inserted into the body of the wearer for drug delivery. In an exemplary embodiment, the needle 86 is a 31G needle. FIG. 27 is a perspective diagram of a cannula needle 300 to deliver insulin or the like in the injection port 84. For example, the needle 300 can be a 90-degree angle stainless steel cannula needle to deliver the insulin with a 4 mm length.

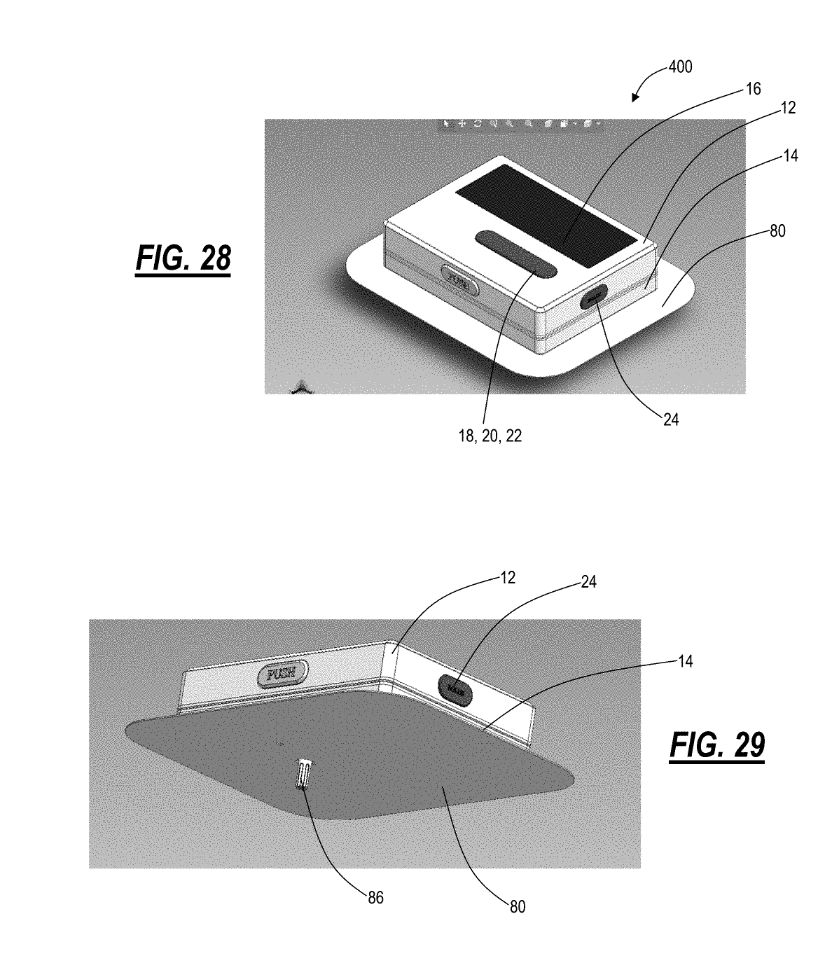

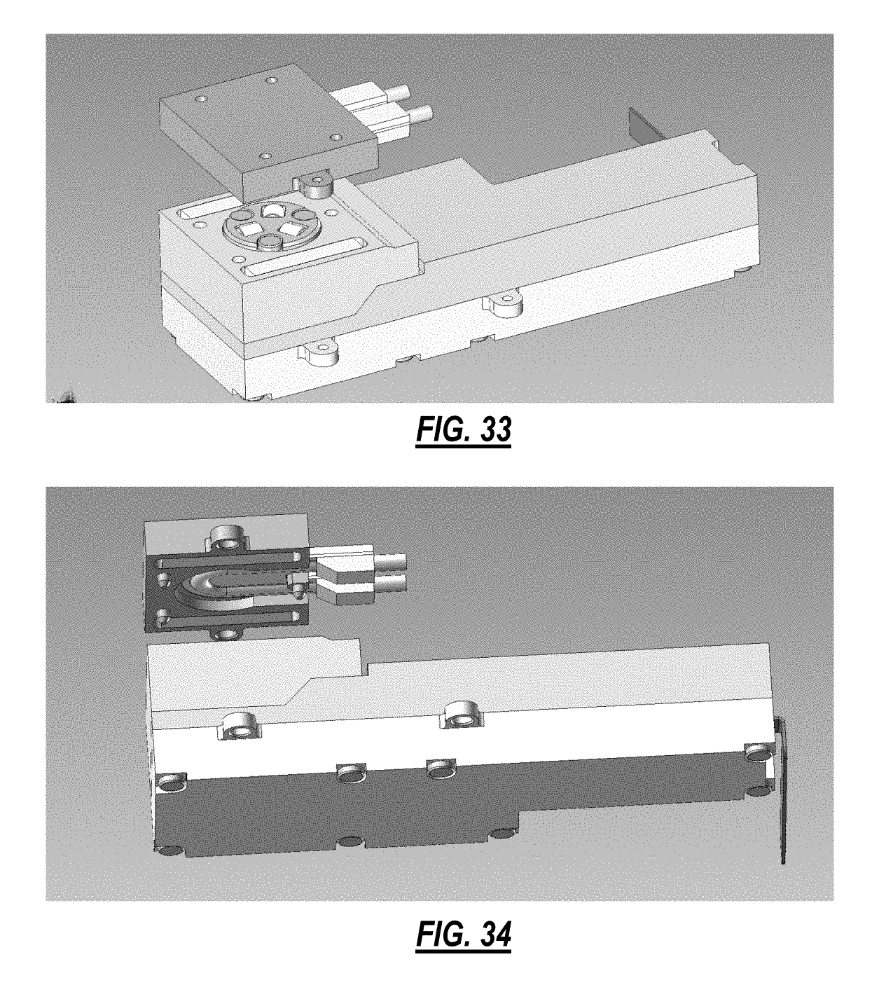

FIGS. 28-32 are perspective diagrams of another exemplary embodiment of a wearable insulin pump 400. The wearable insulin pump 400 has similar components and operation as the wearable insulin pumps 10, 100 with a slightly different form factor. FIGS. 28 and 29 are perspective diagrams (different views) of the wearable insulin pump 400 including the with the pump component 12 connected to the patch component 14 and the adhesive pad 80. The wearable insulin pump 400 includes the display 16, the bolus button 24, and an input bar 18, 20, 22.

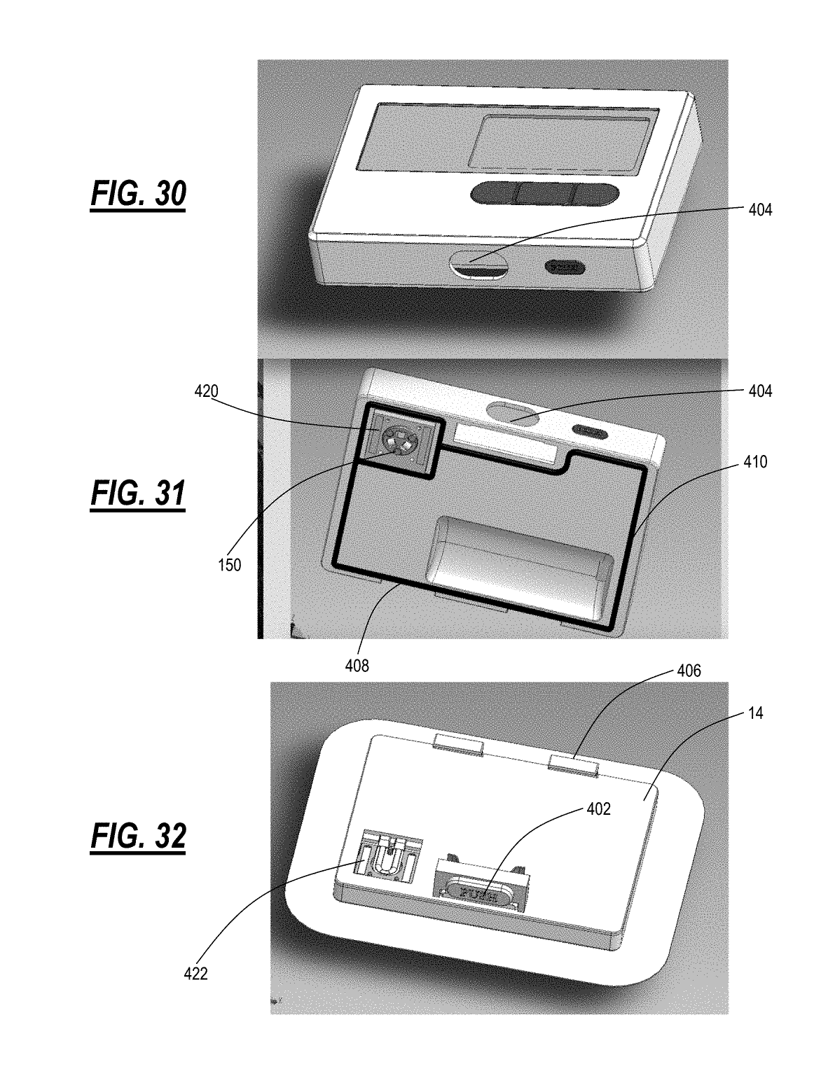

FIGS. 30-32 illustrate connectivity between the pump component 12 and the patch component 14 in the wearable insulin pump 400. The patch component 14 includes a button 402 which can selectively engage an opening 404 the pump component 12 and one or more notches 406 that also engage a slot 408 in the pump component 12. The pump component 12 can include a waterproof seal 410 which engages the patch component 14 for water sealing. The water sealing enables the wearable insulin pump 400 (as well as the wearable insulin pump 10, 100) to be worn while swimming, showering, exercising, etc.

The pump component 12 includes the pump 150 which can include its own waterproof seal 410. The underside of the pump component 12 can include magnets 420 next to the pump 150. The magnets 420 can engage corresponding magnets 422 on the patch component 14 which align the pump 150 to the tube 152.

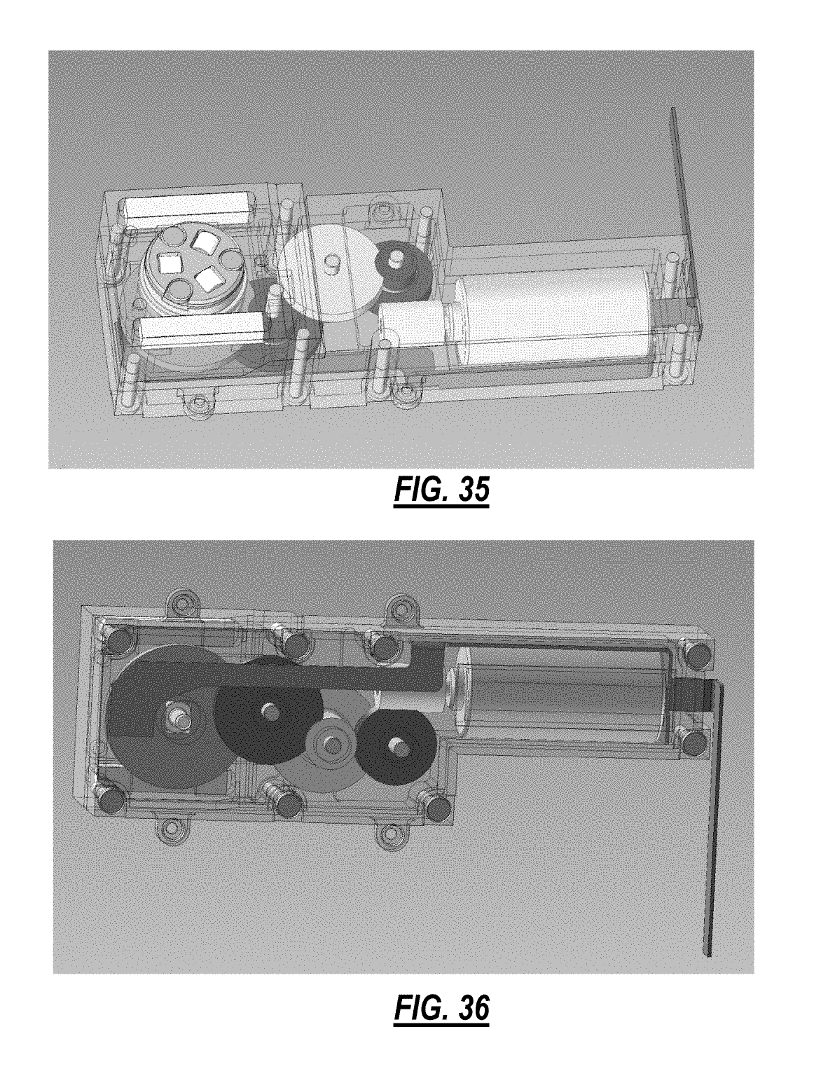

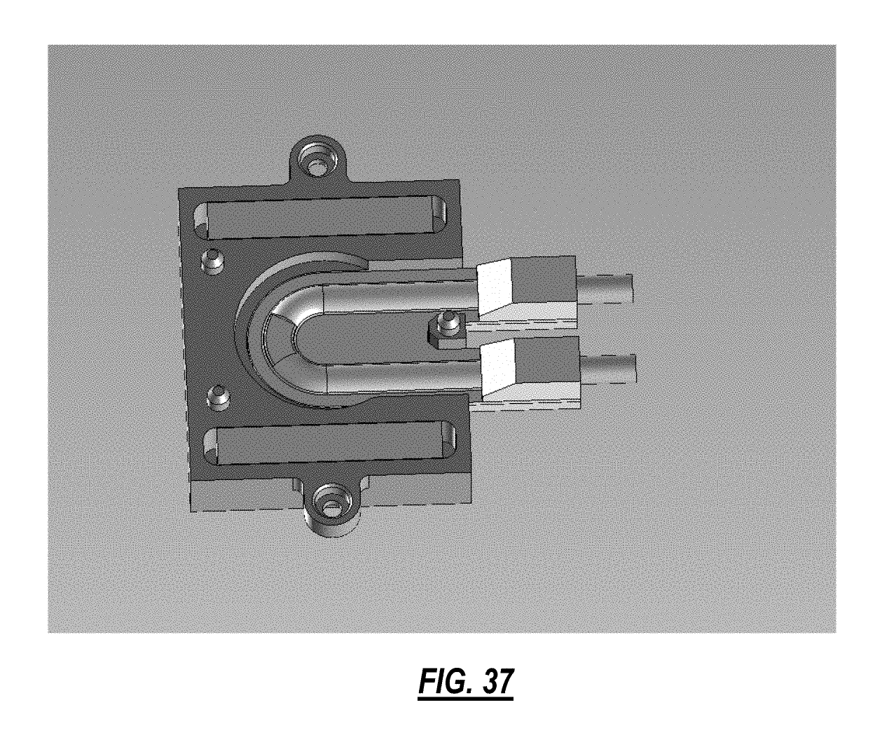

FIGS. 33-37 are perspective diagrams of the pump 150 on the pump component 12, the tube 152 on the patch component 14, and the motor 32. Again, the pump 150 uses peristaltic pump technology to pump insulin through the tube 152. This peristaltic pump technology uses the motor 32 to drive the rotor with the rollers which squeeze the tube 152 to deliver the insulin from the reservoir. The relationship between the pump 150 and the tube can be on the sides, from the top (illustrated here), or from the bottom.

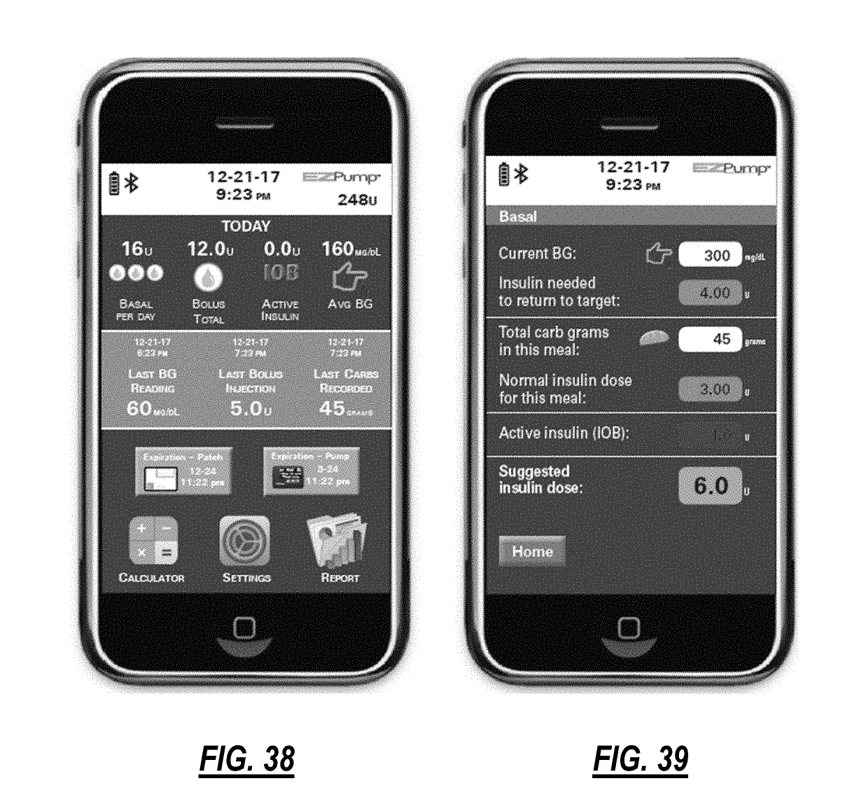

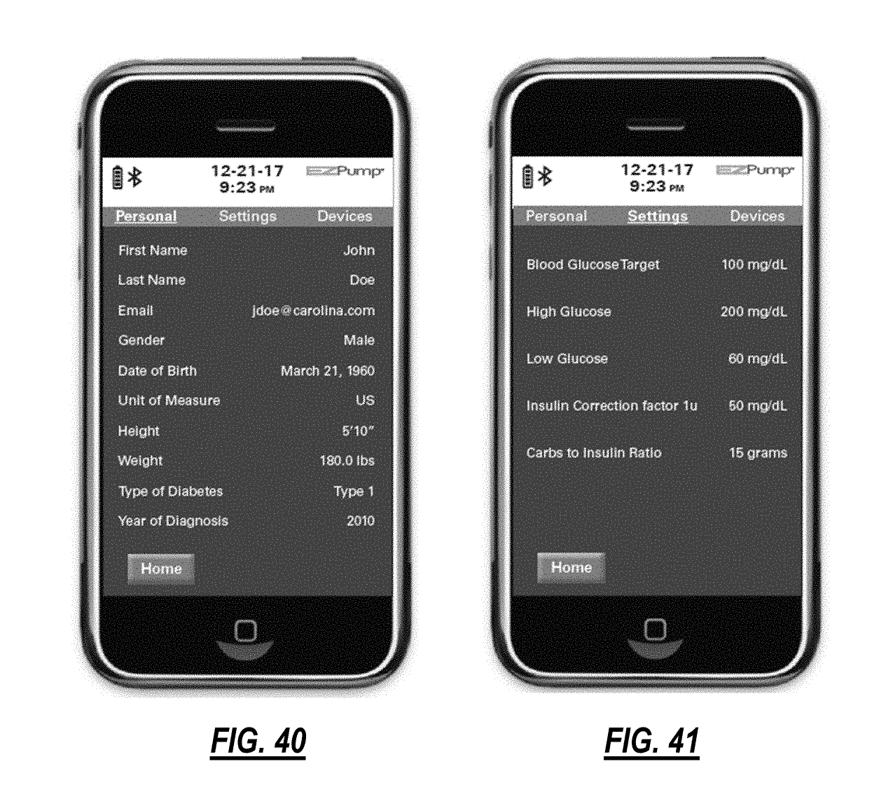

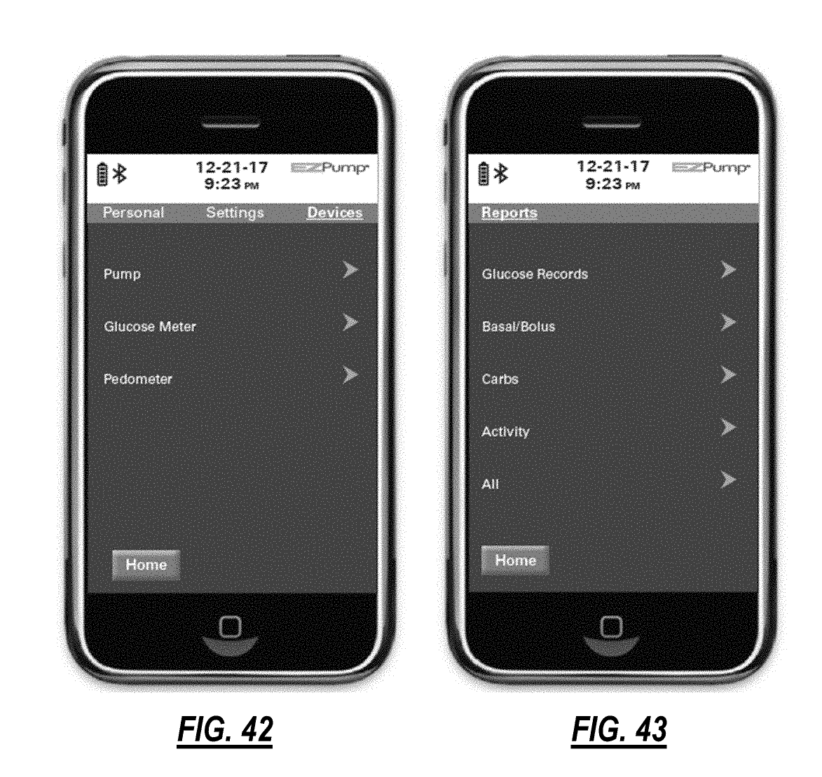

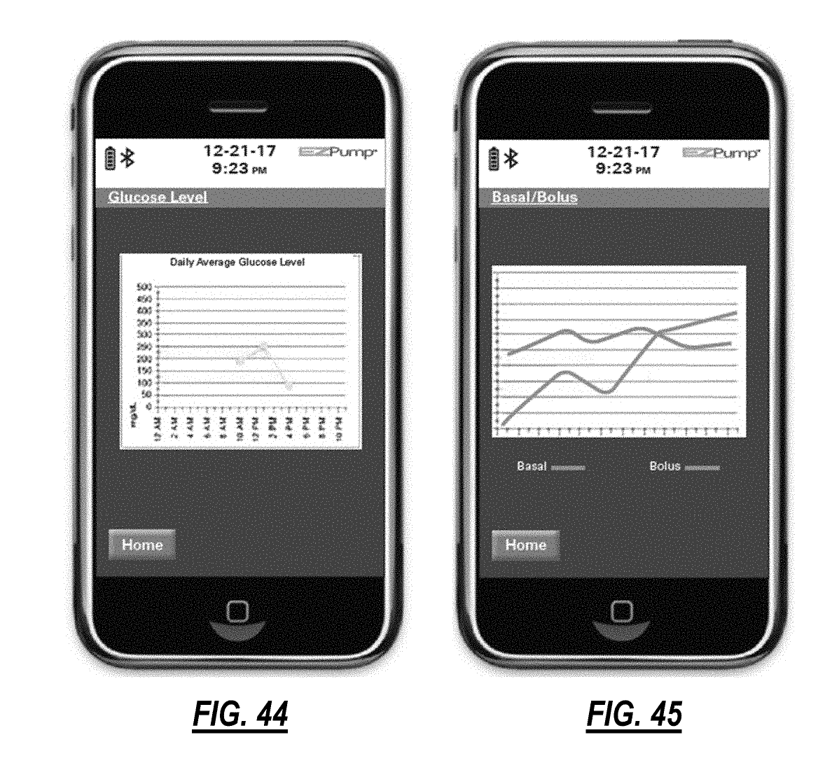

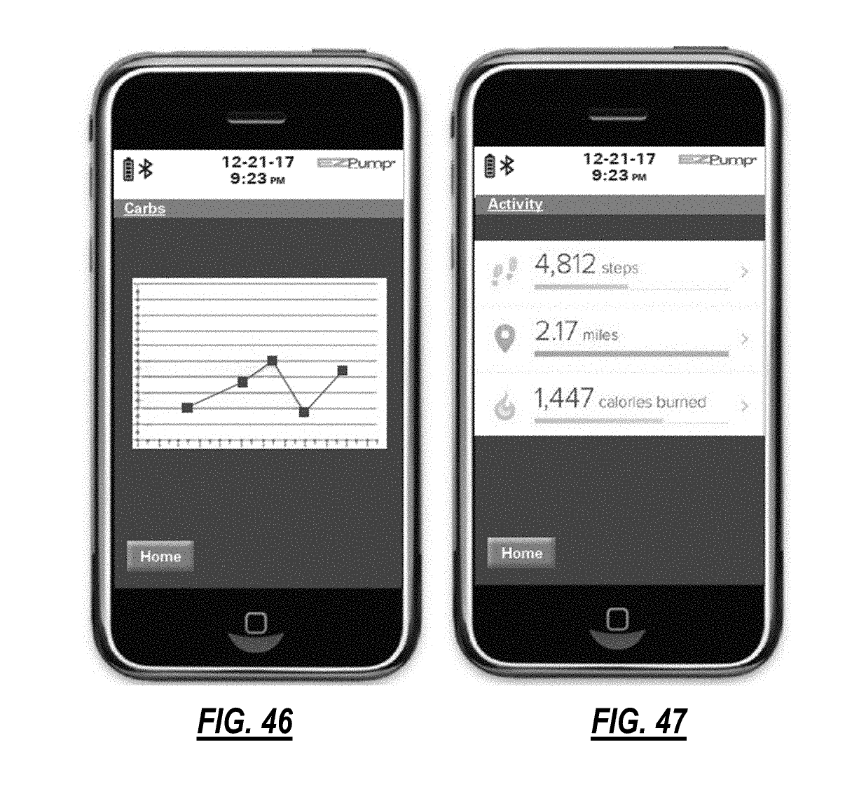

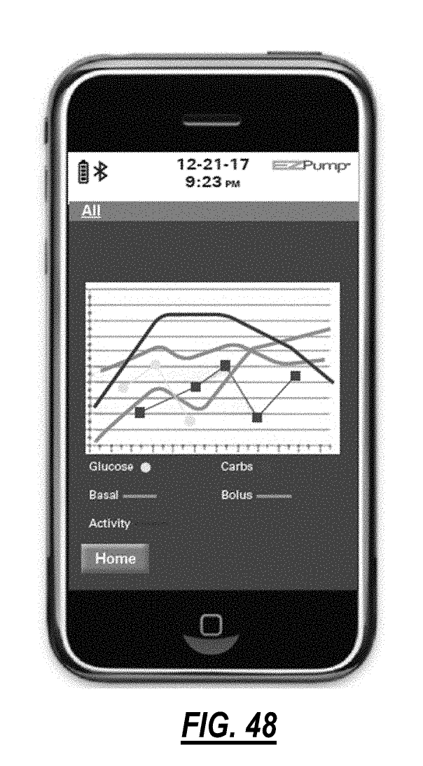

FIGS. 38-48 are screen shots of a mobile application associated with the wearable insulin pump.

It will be appreciated that some exemplary embodiments described herein may include one or more generic or specialized processors (“one or more processors”) such as microprocessors; Central Processing Units (CPUs); Digital Signal Processors (DSPs): customized processors such as Network Processors (NPs) or Network Processing Units (NPUs), Graphics Processing Units (GPUs), or the like; Field Programmable Gate Arrays (FPGAs); and the like along with unique stored program instructions (including both software and firmware) for control thereof to implement, in conjunction with certain non-processor circuits, some, most, or all of the functions of the methods and/or systems described herein. Alternatively, some or all functions may be implemented by a state machine that has no stored program instructions, or in one or more Application Specific Integrated Circuits (ASICs), in which each function or some combinations of certain of the functions are implemented as custom logic or circuitry. Of course, a combination of the aforementioned approaches may be used. For some of the exemplary embodiments described herein, a corresponding device in hardware and optionally with software, firmware, and a combination thereof can be referred to as “circuitry configured or adapted to,” “logic configured or adapted to,” etc. perform a set of operations, steps, methods, processes, algorithms, functions, techniques, etc. on digital and/or analog signals as described herein for the various exemplary embodiments.

Moreover, some exemplary embodiments may include a non-transitory computer-readable storage medium having computer readable code stored thereon for programming a computer, server, appliance, device, processor, circuit, etc. each of which may include a processor to perform functions as described and claimed herein. Examples of such computer-readable storage mediums include, but are not limited to, a hard disk, an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory), Flash memory, and the like. When stored in the non-transitory computer readable medium, software can include instructions executable by a processor or device (e.g., any type of programmable circuitry or logic) that, in response to such execution, cause a processor or the device to perform a set of operations, steps, methods, processes, algorithms, functions, techniques, etc. as described herein for the various exemplary embodiments.

Although the present disclosure has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present disclosure, are contemplated thereby, and are intended to be covered by the following claims.

What is claimed is:

1. A wearable insulin pump, comprising:
a patch component, wherein the patch component comprises an adhesive pad, a fixed needle, a flexible tube, and collapsible reservoir with an injection port connected to the flexible tube and storing insulin; and
a pump component, wherein the pump component comprises a peristaltic pump configured to pump the insulin from the flexible tube to the fixed needle and a controller for control thereof.

2. The wearable insulin pump of claim 1, wherein the patch component is in a first housing and the pump component is in a second housing, wherein the first housing and the second housing are selectively connected to one another, and wherein the pump component is reusable with one or more pump components.

3. The wearable insulin pump of claim 1, wherein the patch component is attached to a user and the fixed needle inserted to delivery insulin, with the pump component attached to the patch component.

4. The wearable insulin pump of claim 1, wherein the peristaltic pump operates by pushing the insulin through the silicon tube via a rotor and one or more rollers.

5. The wearable insulin pump of claim 4, wherein the peristaltic pump is operated by a motor comprising one of i) a worm gear, rollers, and rotors with a motor.

6. The wearable insulin pump of claim 5, wherein the motor is a flat or cylinder and compact, wherein the patch component and the pump component are less than 0.5 inches in depth.

7. The wearable insulin pump of claim 4, wherein the motor comprises a rotatable shaft that connects to the rotor on the patch component.

8. The wearable insulin pump of claim 4, wherein the one or more rollers are disposed on the rotor.

9. The wearable insulin pump of claim 1, wherein the patch component further comprises one or more batteries which provide power to the pump component.

10. The wearable insulin pump of claim 1, wherein the pump component further comprises a display screen for settings and viewing results.

11. The wearable insulin pump of claim 1, wherein the pump component further comprises a vibration unit configured to cause vibration/alarm alerts to a wearer.

12. The wearable insulin pump of claim 1, wherein the collapsible reservoir comprises an injection port where a user injects the insulin.

13. The wearable insulin pump of claim 1, wherein the pump component supports a bolus delivery rate and a basal delivery rate.

14. The wearable insulin pump of claim 1, wherein the pump component further comprises a bolus button for provide a bolus delivery in addition to a continuous basal delivery.

15. The wearable insulin pump of claim 1, wherein the pump component further comprises a plurality of buttons for configuration and control.

16. The wearable insulin pump of claim 2, wherein the pump component is usable for about 90 days and wherein the patch component is usable for about 3 days.

17. The wearable insulin pump of claim 2, wherein the first housing comprises a plurality of openings and a button for connection with a plurality of notches and a snap on the second housing.

18. The wearable insulin pump of claim 2, wherein the first housing and the second housing are sealed with one another via a sealing gasket for waterproofing.

19. A wearable insulin pump kit, comprising:
a plurality of first housings each comprising a patch component comprising an adhesive pad, a fixed needle, a flexible tube, and a collapsible reservoir with an injection port connected to the flexible tube and storing insulin; and
a second housing comprising a pump component comprising a peristaltic pump configured to pump the insulin from the flexible tube to the fixed needle and a controller for control thereof;
wherein the second housing is selectively connectable to each of the plurality of first housings and reusable with each of the plurality of first housings.

20. A method providing a wearable insulin pump, comprising:
providing a patch component in a first housing, wherein the patch component comprises an adhesive pad, a fixed needle, a flexible tube, and a collapsible reservoir with an injection port connected to the flexible tube and storing insulin; and
providing a pump component in a second housing, wherein the pump component comprises a peristaltic pump configured to pump the insulin from the flexible tube to the fixed needle and a controller for control thereof;
wherein the first housing and the second housing are selectively connected to one another, and wherein the pump component is reusable with one or more pump components.
